(19) United States
(12) Patent Application Publication
(10) Pub. No.: US 2023/0106452 A1
(43) Pub. Date: Apr. 6, 2023

(54) PHANTOM APPARATUS AND METHODS THEREFOR

(71) Applicant: Calimetrix LLC, Middleton, WI (US)

(72) Inventors: Jean H Brittain, Middleton, WI (US); David R Rutkowski, Seattle, WA (US)

(21) Appl. No.: 17/959,830

(22) Filed: Oct. 4, 2022

Related U.S. Application Data
(60) Provisional application No. 63/251,908, filed on Oct. 4, 2021.

(51) Int. Cl. G01R 33/58; A61B 5/055; G09B 23/28

(52) U.S. Cl. CPC G01R 33/58; A61B 5/055; G09B 23/286

(57) ABSTRACT

Aspects of the disclosure are directed to a phantom apparatus, such as may be used in MRI imaging. As may be implemented with a particular embodiment, such an apparatus may include a first tissue-mimicking region having a first tissue property, and at least one additional tissue-mimicking region, including a second tissue-mimicking region having a second tissue property that is different than the first tissue property. The second tissue-mimicking region is stacked on the first tissue-mimicking region.

### BACKGROUND

For many imaging and diagnostic type equipment, it can be desirable to utilize testing componentry to assess the equipment operation and/or to assess the capability of users of the equipment. One such approach involves the utilization of a phantom (test object) designed to be imaged and/or scanned, for testing of equipment and software carrying out the imaging and/or scanning. For instance, phantoms may be used with magnetic resonance (MR) (e.g., magnetic resonance imaging (MRI) and magnetic resonance elastography (MRE)). Tissue-mimicking phantoms may serve as reference standards for assessing relevant equipment and software, and/or for assessing the manner in which such equipment and software are being used.

While useful, phantoms may be limited in their function, and may need to be tailored to the particular application in which they are utilized. These and other matters have presented challenges to the design and implementation of phantoms for a variety of applications.

### SUMMARY

Accordingly, various embodiments are directed to phantoms, their application and their manufacture, as may address the issues noted above. A particular embodiment is directed to a phantom apparatus having tissue-mimicking regions, each region respectively having one or more properties that are different from properties exhibited by another one of the regions.

A more particular embodiment is directed to a phantom apparatus having tissue-mimicking regions, each region respectively having a tissue property that is different from a tissue property exhibited by another one of the regions. Such tissue properties may involve mechanical stiffness, properties mimicking fat content, and/or others. Another particular embodiment is directed to a phantom having tissue-mimicking regions, each region respectively having mechanical stiffness and/or fat content and/or relaxation properties that are different than mechanical stiffness and/or fat content and/or relaxation properties exhibited by another one of the regions.

Another embodiment is directed to a phantom apparatus comprising a first tissue-mimicking region having a first tissue property and at least one additional tissue-mimicking region, including a second tissue-mimicking region having a second tissue property that is different than the first tissue property. The second tissue-mimicking region may be stacked on the first tissue-mimicking region. In some implementations, an external surface of a first tissue-mimicking region interfaces with an external surface of a second tissue-mimicking region to form a contiguous portion of an outer surface of the tissue-mimicking regions. In these contexts, the respective regions may have different values of the same property, different properties, or combinations of multiple properties in each layer with at least one property changing between the layers.

Various embodiments are directed to methods of making a phantom as characterized herein, and to methods of using such a phantom. For instance, a particular embodiment is directed to analyzing an imaging system by imaging a phantom having tissue-mimicking regions, each region respectively having a tissue property that is different than a tissue property exhibited by another one of the regions.

Additional embodiments are directed designs that facilitate efficient and repeatable setup in the clinical environment. A stackable system utilizes complementary components that facilitate magnetic field homogeneity within a magnetic resonance environment.

The above discussion/summary is not intended to describe each embodiment or every implementation of the present disclosure. The figures and detailed description that follow also exemplify various embodiments.

### BRIEF DESCRIPTION OF FIGURES

Various example embodiments may be more completely understood in consideration of the following detailed description and in connection with the accompanying drawings, in which:

FIGS. 1A and 1B show perspective and cross-sectional views of a vertically-stacked cylindrical phantom apparatus, in accordance with one or more embodiments;

FIGS. 2A and 2B show perspective and cross-sectional views of a split cylindrical phantom apparatus, in accordance with one or more embodiments;

FIGS. 3A and 3B show perspective and cross-sectional views of a cylindrical core phantom apparatus, in accordance with one or more embodiments;

FIGS. 4A and 4B show perspective and cross-sectional views of a spherical phantom apparatus, in accordance with one or more embodiments;

FIGS. 5A and 5B show coronal and exploded isometric views of a composite pill-shaped phantom, in accordance with one or more embodiments;

FIG. 6 shows a stacked slab or pad-type phantom apparatus, in accordance with one or more embodiments;

FIG. 7 shows a stacked slab or pad-type phantom apparatus, in accordance with one or more embodiments; and

FIG. 8 shows a stacked slab or pad-type phantom apparatus, in accordance with one or more embodiments.

While various embodiments discussed herein are amenable to modifications and alternative forms, aspects thereof have been shown by way of example in the drawings and will be described in detail. It should be understood, however, that the intention is not to limit the invention to the particular embodiments described. On the contrary, the intention is to cover all modifications, equivalents, and alternatives falling within the scope of the disclosure including aspects defined in the claims.

### DETAILED DESCRIPTION

Aspects of the present disclosure are believed to be applicable to a variety of different types of articles of manufacture, apparatuses, systems and methods involving phantoms. In certain implementations, aspects of the present disclosure have been shown to be beneficial when used in the context of phantoms having regions of differing characteristics. While not necessarily so limited, various aspects may be appreciated through a discussion of examples using such exemplary contexts.

Various embodiments are directed to a phantom having tissue stiffness compartment configurations that facilitate high quality quantitative measurements over a range of human in-vivo tissue stiffness. This may involve quantitative MR elastography (MRE) phantom design and production. Related embodiments are directed to a phantom having stacked regions of differing characteristics that facilitate testing.

Particular embodiments are directed toward an MRE phantom that enables an MR user to measure the stiffness of multiple materials with different known stiffness values in a single MRE scan. The spatial extent occupied by components of different material stiffness is selected and positioned relative to other materials in the phantom in a way that facilitates accurate measurement of the stiffness of each material, for instance using an MRE system that operates with a specific excitation wavelength and frequency. The overall size of the MRE phantom may be sufficiently small to enable easy positioning of the phantom in the MR system and relative to MR elastography hardware being utilized.

Another embodiment is directed to an apparatus having tissue-mimicking material configured to simultaneously mimic both mechanical stiffness and magnetic resonance imaging (MRI) proton density fat fraction.

A phantom of a particular embodiment has stacked regions of different tissue properties, such as stiffness. The stacked regions may facilitate utilization of a set compartment depth and/or diameter to ensure quantitative accuracy for a given compartment stiffness and excitation frequency. The stiffness or other tissue property of each layer may be measured using a multi-slice MRE approach, where each slice is prescribed to lie within each layer.

Another embodiment is directed to a phantom having stacked regions of different properties, as may include layers of different materials and/or separate compartments in which each compartment holds a different type of layer of tissue-mimicking materials. This approach may enable the use of materials that do not have a thermoset property (e.g., as described herein), as may include liquids and/or gels. In various contexts, such different properties may involve different tissue properties in which each layer and/or compartment has a different value of tissue properties or a combination of properties.

Respective surface portions of each of such tissue-mimicking regions may collectively form an outer surface of a portion of the phantom apparatus that consists of the tissue-mimicking regions, each tissue-mimicking region having such a surface portion that is contiguous with such a surface portion of another of the tissue-mimicking regions. For instance, the phantom may have an outer shell that encompasses the tissue-mimicking regions, which collectively form a set of such regions having an outer surface within the shell.

In some embodiments, a stacked phantom is manufactured with thermoset polymers, cross-lined polymers, or a gel that has a melting point higher than its gel point by pouring one layer at a time, allowing the layer to cool, and then pouring an additional layer. Any number of layers may be formed, as may be tailored to MR coil size and MRE wavelength. Because thermosets will not melt after initial cure, potentially negative effects of adding hot liquid of subsequently poured layers may be mitigated.

A phantom as characterized herein may have a shell having an outer surface and an inner surface within which the tissue-mimicking regions reside. Respective surface portions of each of the tissue-mimicking regions may collectively form an outer surface of the tissue-mimicking regions having a shape that follows a shape of the inner surface of the shell.

Various embodiments are directed to materials that mimic a type of MR tissue property, such as proton density fat fraction (PDFF), R2*, T1, T2, Magnetization Transfer, T1rho. As such, more than one substance with different properties in different sections (e.g., layers) within a single container (e.g., a cylindrical, spherical, or pill shaped container) or a single container with multiple compartments may be implemented with a variety of materials. Such compartments may be attached, such as by locking together, and may facilitate the addition or removal of compartments to suit particular applications. Different sections/layers may mimic different tissue properties, different values of the same tissue property, and/or multiple tissue properties that are different than the properties of other sections/layers. Further, the tissue mimicking material may extend such that a corresponding compartment containing the material may be adjacent to or the same as the housing wall in at least one dimension.

Certain embodiments are directed to combined elastography/PDFF phantoms configured to represent various levels of PDFF and tissue stiffness, which may also exhibit various MR relaxation parameters.

Various phantoms may simultaneously mimic tissue stiffness, fat-fraction and/or MRI relaxation properties. Water-based gelatins and/or polymeric gels can be emulsified with oil and surfactant to create a substance that mimics the proton density fat fraction (PDFF) of human tissue. The gel concentration of these same water-based materials can be modified to modulate material mechanical stiffness. By modulating the oil percentage and/or the base-material gel percentage in the water-oil emulsion, a full physiological range of fat fractions (0-100%) and/or tissue stiffness (0-12 kPa in the liver) can be represented by these emulsions. Furthermore, a variety of salts or other substances including iron-containing particles can be added to the gel to modulate MRI relaxation times (T1, T2, T2*).

Phantoms thus may be implemented with multiple layers, sections, or compartments that contain different gels, liquids or other materials that simultaneously mimic different combinations of tissue properties including tissue stiffness, fat-fraction, and/or one or more MRI relaxation properties. Multiple gels that simultaneously mimic different combinations of MRI properties (e.g., varying stiffness, fat-fraction, and/or a relaxation property compared to other gels in the same phantom or phantom set) can also be stacked or otherwise placed in adjacent sections or compartments as described herein.

A phantom apparatus as characterized herein may have tissue-mimicking regions having varied tissue properties, such as varied mechanical stiffness. The regions may be arranged in cylindrical, spherical, cylindrical with rounded/hemispherical ends (e.g., as may be referred to as pill-shaped), stacked, layered slab, concentric and/or centrally split configurations. The phantom may include a housing with the regions and/or phantom housing having a circular cross-section and/or cylindrical outer shell that facilitates transmission of concentric mechanical waves throughout the tissue-mimicking region.

In a particular embodiment, a phantom apparatus is arranged in a slab or pad type arrangement, with two or more stacked layers. This may facilitate use with a patient lying upon the phantom. For instance, an MRI bed may include a pad having stacked layers as characterized herein, operable as a phantom. The pad may be utilized while a patient is lying upon the pad, or at times when no patient is present.

The regions may be provided in a variety of manners. In some embodiments, each region is a compartment, with one or more compartments being stacked on a first compartment. Each region may have a different type of material.

One or more regions may include a thermoset material, cross-linked polymeric material, or gel-based material that has a melting point higher than its gel point. Such a region may be configured to set in response to being heated and subsequently cooled. This may facilitate the application of another type of material onto the preceding material while mitigating changes in properties of the preceding material.

The spatial extent occupied by each region and materials therein may be selected and positioned relative to the other regions to facilitate measurement of the stiffness of the material in the region, based on one or more characteristics of an imaging system. For instance, the regions may be defined to facilitate testing characteristics relating to excitation wavelength (e.g., mechanical/MRE), frequency, passive driver type and format, or a combination thereof.

Another embodiment is directed to a phantom having tissue-mimicking regions respectively having mechanical stiffness and/or fat content and/or relaxation properties that are different than mechanical stiffness and/or fat content and/or relaxation properties exhibited by another one of the regions. Each region may be a compartment representing mechanical stiffness and/or MRI proton density fat fraction and/or MRI relaxation properties, with one or more compartments being stacked on a first compartment. One or more of the regions may include an elastic material emulsified with oil and a surfactant to simultaneously modulate MRE stiffness and MRI PDFF measurements. Material stiffness may be modulated across multiple compartments in a range of 1-30 kPA, and MRI PDFF may be modulated across multiple compartments in a range of 0-100% fat fraction.

One or more regions may include an elastic material emulsified with oil and a surfactant, with salt ions, or with other substances including iron-containing particles added for modulation of MRI relaxation times. These may correspond to times relating to one of the following conditions, or a combination thereof, including tissue fat content, tissue fibrosis, tissue fluid content, and tissue iron concentration.

A method for analyzing an imaging system may be carried out using a phantom having tissue-mimicking regions as characterized herein. Each region may respectively have tissue properties that are different than such properties exhibited by another one of the regions. The regions may be arranged using one or more of a stacked configuration, a concentric configuration, and a centrally split configuration. The imaging system may be analyzed by generating images and/or generating a measurement of each region Where the regions are stacked on one another, analyzing the imaging system may include assessing mechanical stiffness of each region using a multi-slice magnetic resonance elastography (MRE) approach in which one or more slice(s) lies in a stacked region that is different than a stacked region in which other ones of the slices lie.

Another embodiment is directed to a method of manufacturing a phantom. A phantom having tissue-mimicking regions is formed such that each region respectively exhibits tissue property that is different than a tissue property exhibited by another one of the regions. The regions may be formed in one or more of a stacked configuration, a concentric configuration, and a centrally split configuration, or a combination thereof. Each phantom region may be formed with a spatial extent and materials selected and positioned relative to the other regions to facilitate measurement of the stiffness of the material in the region. This material selection and positioning may be set to facilitate testing imaging system characteristics as may account for one or more of excitation wavelength, frequency, passive driver type and format.

Various embodiments are directed to a phantom having tissue-mimicking regions, each region respectively having mechanical stiffness and/or fat content and/or relaxation properties that are different than mechanical stiffness and/or fat content and/or relaxation properties exhibited by another one of the regions. Each region may be a compartment representing mechanical stiffness and/or MRI proton density fat fraction and/or MRI relaxation properties, with one or more compartments being stacked on a first compartment. The material stiffness may be modulated across multiple compartments in a range of 1-30 kPA, and/or MRI PDFF is modulated across multiple compartments in a range of 0-100% fat fraction.

One or more of the regions may include an elastic material emulsified with oil and a surfactant to simultaneously modulate MRE stiffness and MRI PDFF measurements. One of the regions may include elastic material emulsified with oil and a surfactant, with salt ions and/or other substances including iron-containing particles added for modulation of MRI relaxation times corresponding to times relating to one or more of tissue fat content, tissue fibrosis, tissue fluid content, tissue iron concentration, and a combination thereof.

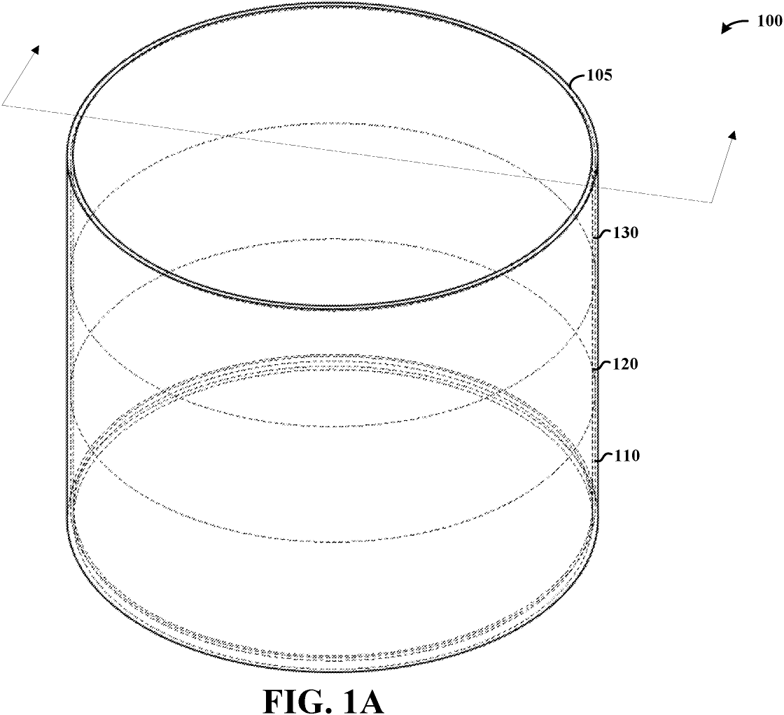

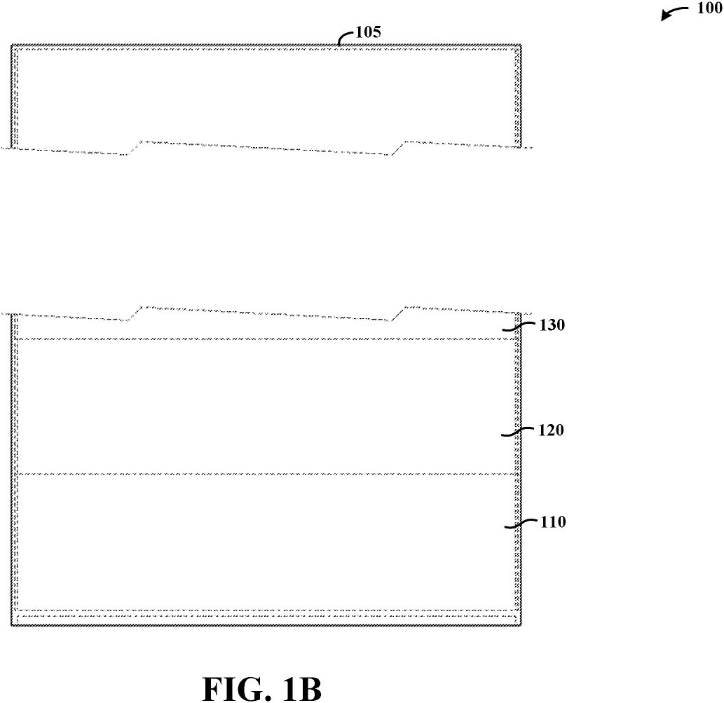

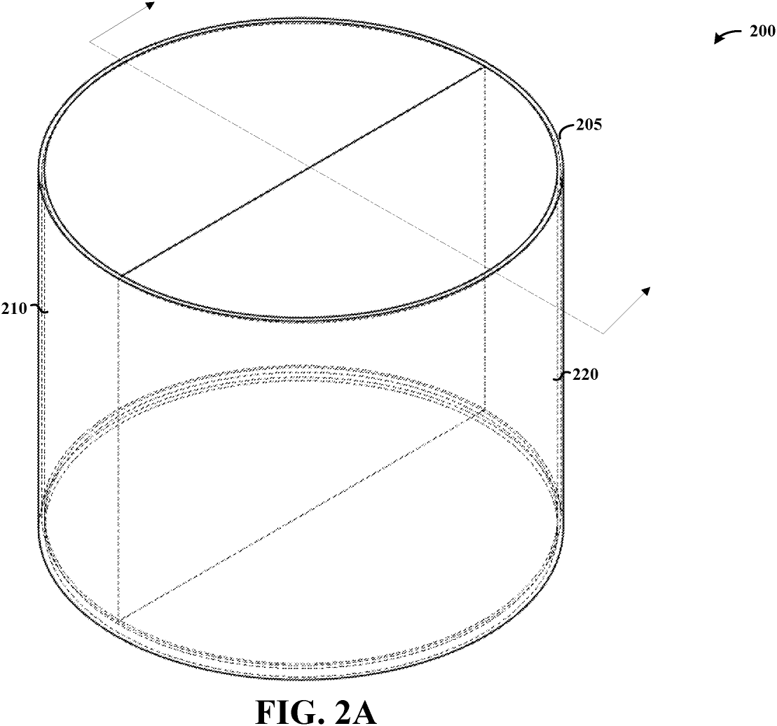

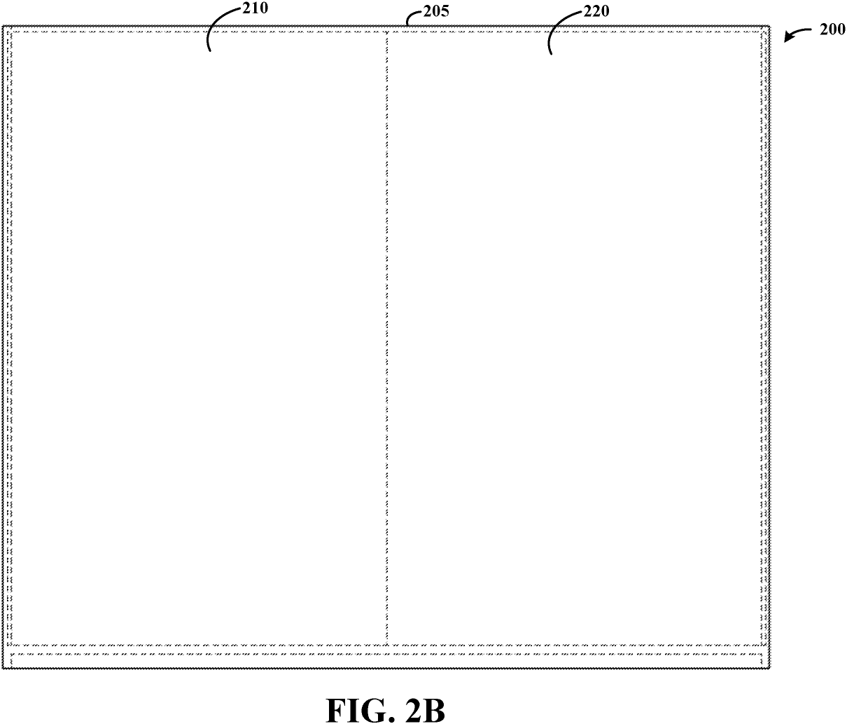

FIGS. 1A and 1B respectively show perspective and cross-sectional views of a stacked cylindrical phantom apparatus 100, in accordance with one or more embodiments. Layers 110, 120 and 130 of different materials are stacked within a housing 105. Fewer or additional layers may be used, for example as represented in FIG. 2B by way of broken lines.

The stacked layers shown in FIG. 1 may be formed using thermoset polymers, cross-lined polymers, a gel that has a melting point higher than its gel point, or other materials. The stacked layers may be formed by pouring one layer at a time. Each poured layer may be allowed to cool prior to pouring an additional layer thereon. Each layer may be a compartment, with the compartment filled with a material having a particular tissue property. The various layers in FIGS. 2A-4B may be formed similarly, and using similar materials.

FIGS. 2A and 2B respectively show perspective and cross-sectional views of a split cylindrical phantom apparatus 200, in accordance with one or more embodiments. Split sections 210 and 220 of different materials are arranged horizontally within a housing 205.

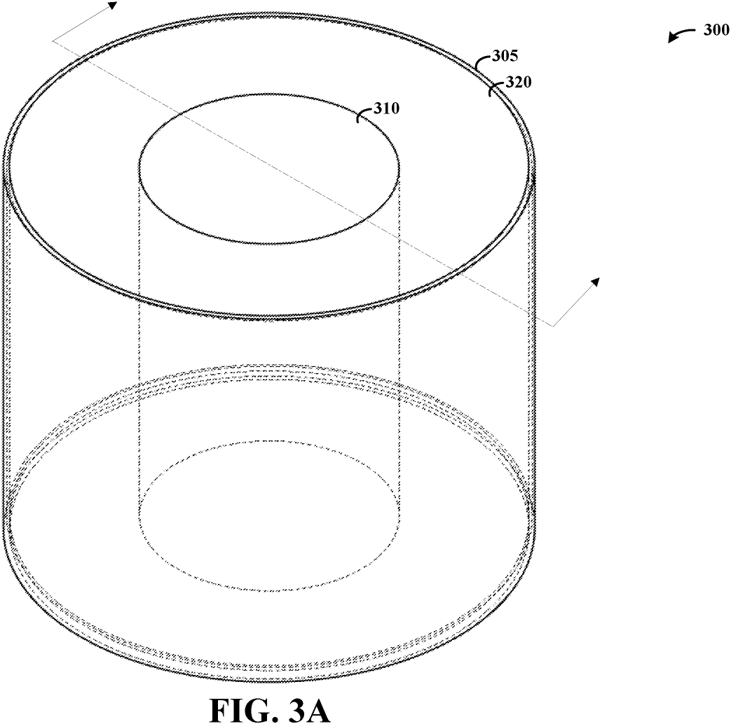

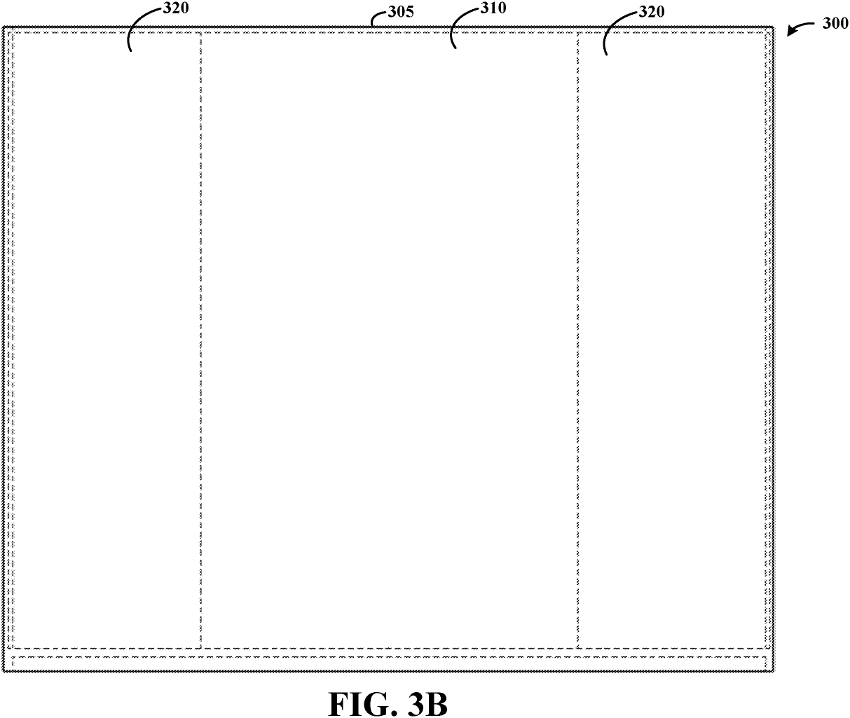

FIGS. 3A and 3B respectively show perspective and cross-sectional views of a cylindrical core phantom apparatus 300, in accordance with one or more embodiments. Concentric core sections 310 and 320 are arranged as shown, within a housing 305.

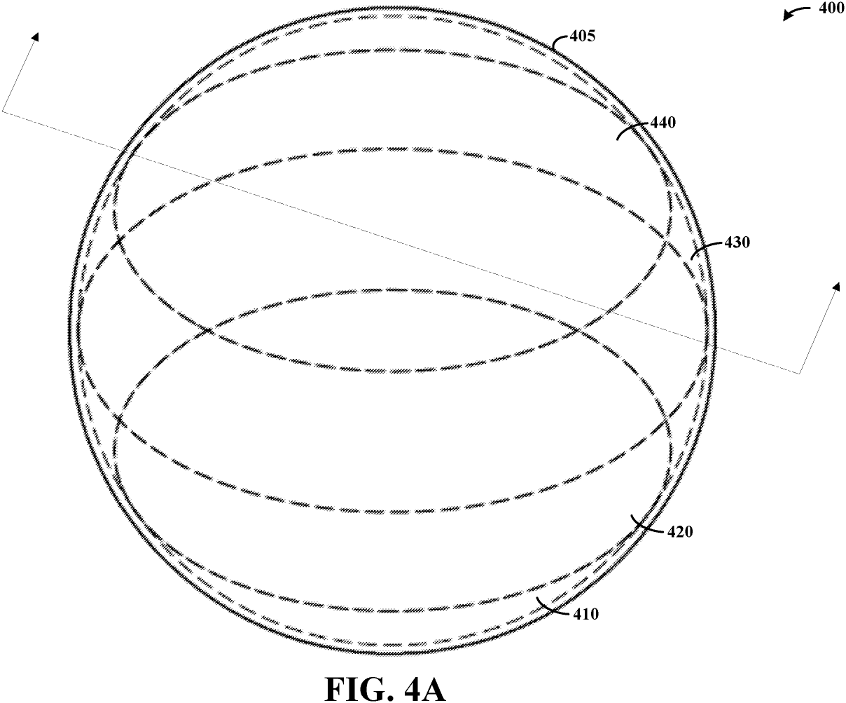

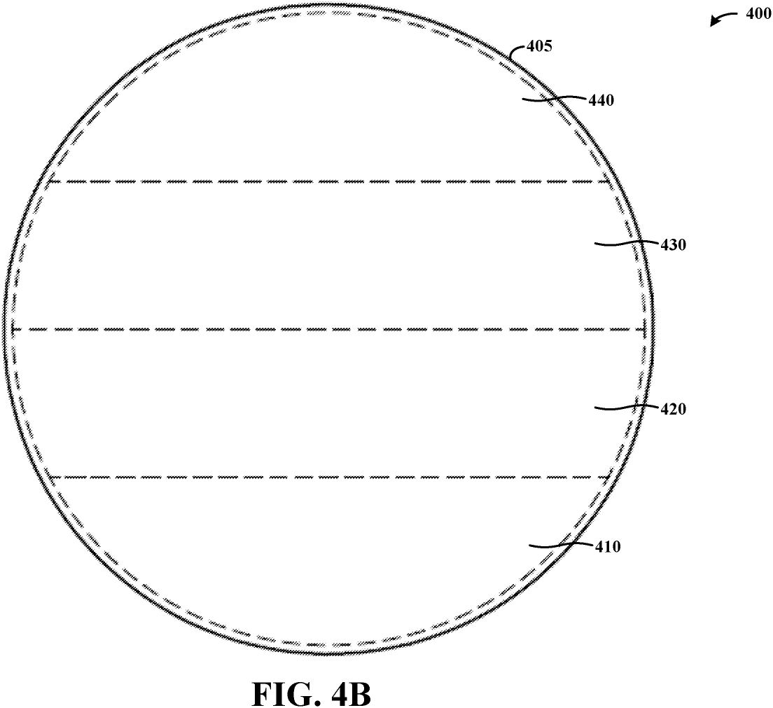

FIGS. 4A and 4B respectively show perspective and cross-sectional views of a spherical phantom apparatus 400, in accordance with one or more embodiments. The apparatus 400 has stacked layers 410, 420, 430 and 440 within a shell 405. The stacked layers 410, 420, 430 and 440 may, for example, be chambers enclosing a tissue mimicking material.

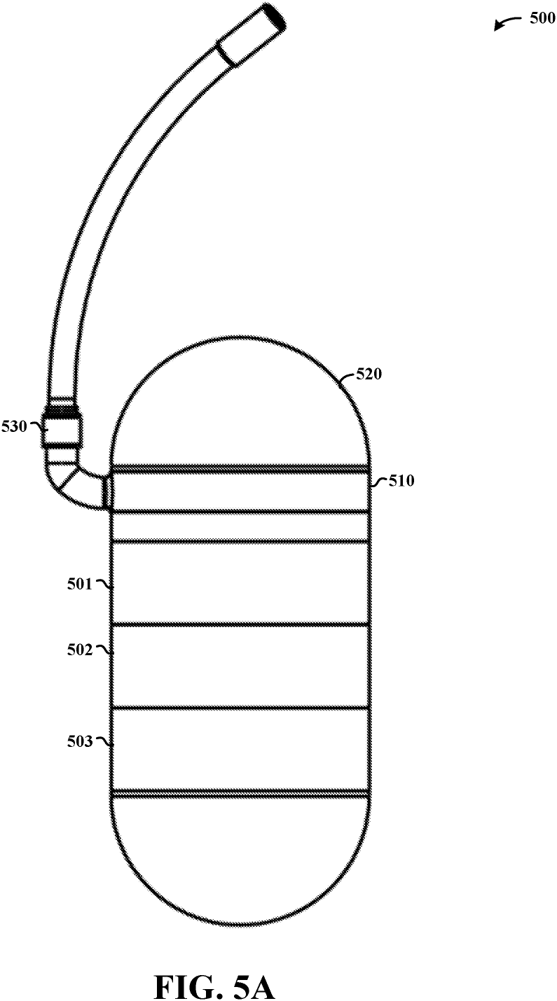

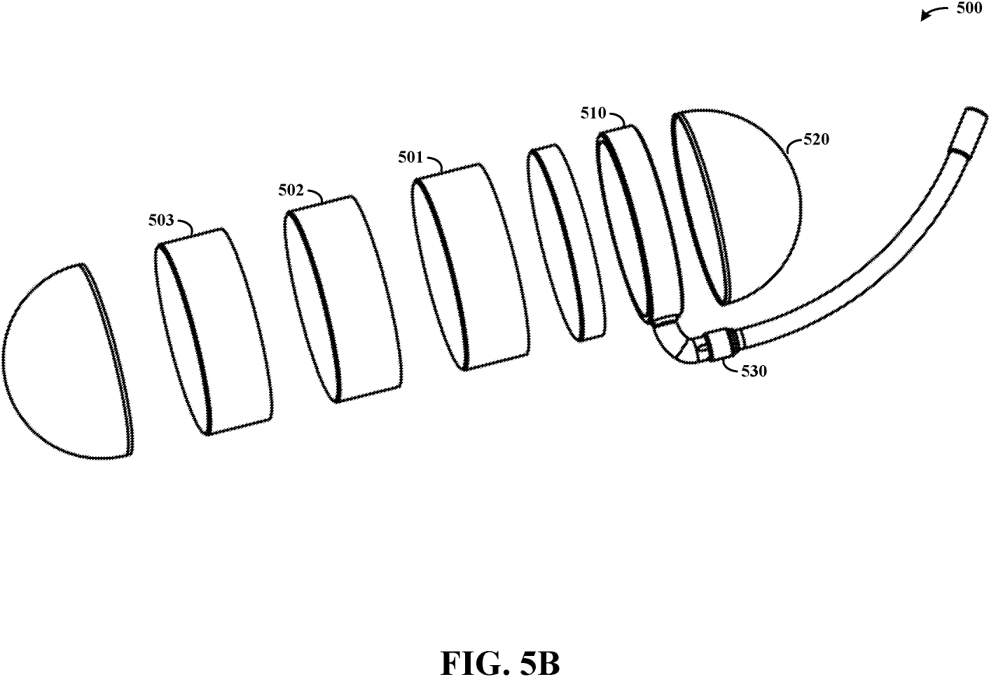

FIGS. 5A and 5B respectively show coronal and exploded isometric views of a composite pill-shaped phantom 500, in accordance with one or more embodiments. The phantom 500 includes stackable, interlocking, cylindrical containers 501, 502 and 503, which can be aligned along an axis. An integrated MRE passive driver 510 may provide mechanical excitation, and a hemispherical end cap may be utilized (see 520) and filled with high-signal material to facilitate magnetic field homogeneity. An air flow attenuator 530 may be utilized for modulating amplitudes of mechanical excitation.

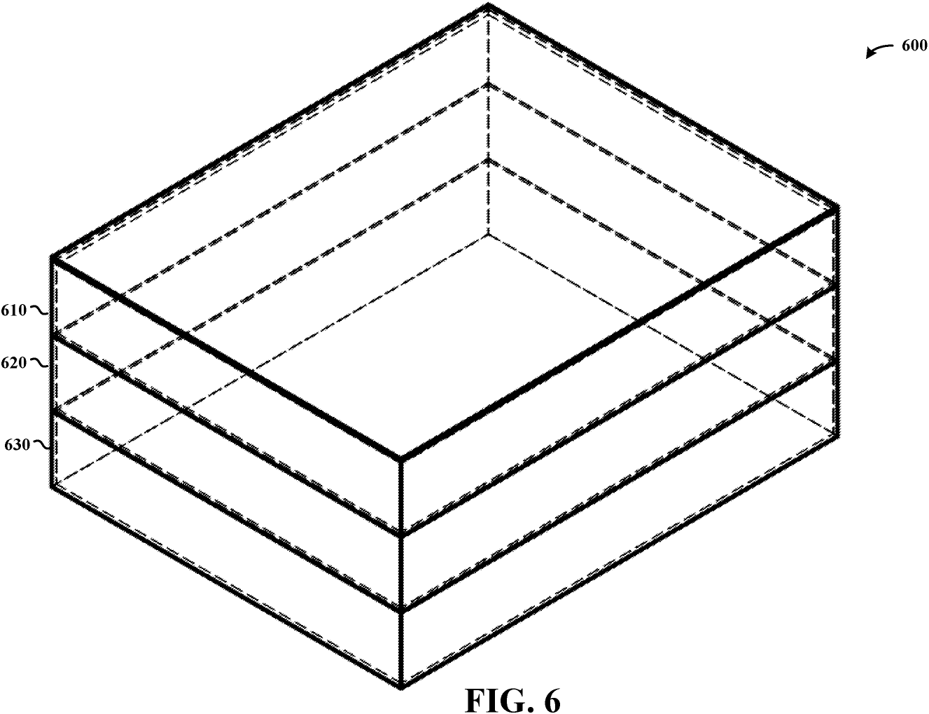

FIG. 6 shows a stacked slab or pad-type phantom apparatus 600, in accordance with one or more embodiments. The apparatus 600 includes three stacked layers 610, 620 and 630, each layer including a tissue-mimicking region. As consistent with other embodiments herein, one or more of the tissue-mimicking regions exhibit different tissue-mimicking properties. These layers may be implemented as compartments with tissue-mimicking regions therein as shown in dashed lines. Alternately, each layer may be of a tissue mimicking material with layer 620 stacked on layer 630, and layer 610 stacked on layer 620. In some implementations, such a stacked slab or pad-type phantom apparatus may be utilized for supporting a patient, such as in the bed of an MRI apparatus.

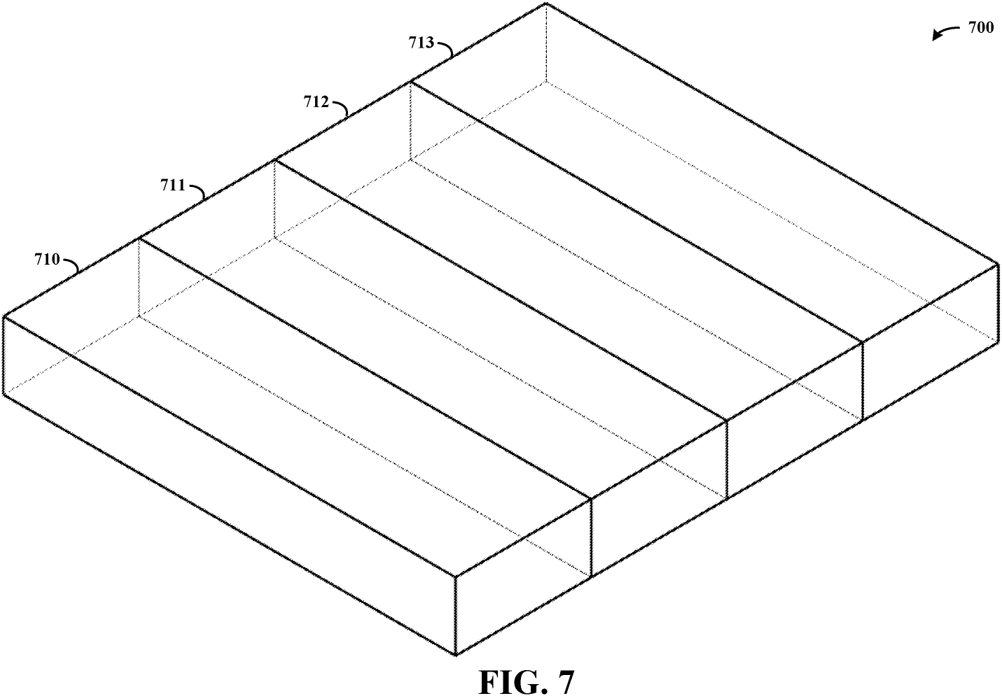

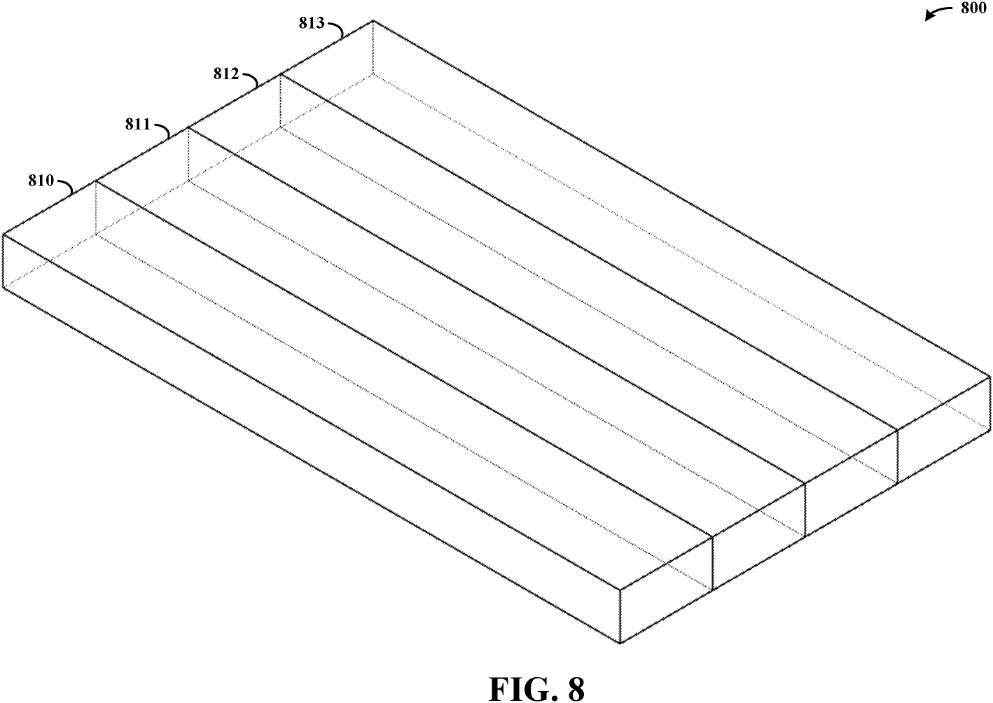

FIGS. 7 and 8 show further stacked slab or pad-type phantom apparatuses, in accordance with one or more embodiments. These apparatuses have stacked tissue-mimicking regions in which one or more regions exhibits different tissue-mimicking properties relative to the other regions. These apparatuses may also be utilized to support a patient as noted above. The apparatus 700 in FIG. 7 includes respective tissue-mimicking regions 710, 711, 712 and 713 stacked horizontally and extending across the width of the apparatus (e.g., a pad). FIG. 8 shows a stacked slab or pad-type phantom apparatus 800 having tissue-mimicking regions 810, 811, 812 and 813 stacked horizontally and extending along the length of the apparatus (e.g., as may also be a pad).

As may be implemented with the apparatus shown in FIGS. 5A and 5B or otherwise, an integrated driver may function similar to passive drivers for MRE active driving systems that may include a tubing connector interface, an air flow cavity, and a thin plastic, semi-flexible membrane that contacts tissue (or a phantom) of interest. This may facilitate the flow of pulses of air from an air supply system, trough the tubing, and into the air cavity. The air pressure fluctuations move the membrane, which transfers mechanical waves through the tissue of interest. This may be integrated with a phantom using a passive driver with a cylindrical shape, so that it can interlock with the tissue mimic stacks and end caps. Such end caps may be plastic hemispherical cavities filled with a liquid or water-based gels (e.g., for an MR signal). They may have interlocking features so that they can connect to the stacks.

An attenuator may include a polymer-based valve that can be adjusted to let varying amount of air pass out of the system. A fully open valve may relieve air pressure from the system and cause lower amplitudes through the driver. A closed valve would not let air escape and increase pressure, and therefore increase mechanical amplitude of the driver. Other valve positions may attenuate airflow and therefore mechanical amplitude at varying levels.

Based upon the above discussion and illustrations, those skilled in the art will readily recognize that various modifications and changes may be made to the various embodiments without strictly following the exemplary embodiments and applications illustrated and described herein. For example, fewer or more layers/compartments may be used, materials of different composition may be used, and a variety of different shapes may be used for both overall phantoms and for regions/layers of different material within those phantoms. In addition, regions as shown may be compartments that contain material of a particular tissue property. Such modifications do not depart from the true spirit and scope of various aspects of the invention, including aspects set forth in the claims.

What is claimed is:

1. A phantom apparatus comprising:
a first tissue-mimicking region having a first tissue property; and
at least one additional tissue-mimicking region, including a second tissue-mimicking region having a second tissue property that is different than the first tissue property, the second tissue-mimicking region being stacked on the first tissue-mimicking region.

2. The apparatus of claim 1, wherein respective surface portions of each of the tissue-mimicking regions collectively form an outer surface of a portion of the phantom apparatus that consists of the tissue-mimicking regions, each tissue-mimicking region having such a surface portion that is contiguous with such a surface portion of another of the tissue-mimicking regions.

3. The apparatus of claim 1, wherein:
the phantom has a shell having an outer surface and an inner surface within which the tissue-mimicking regions reside; and
respective surface portions of each of the tissue-mimicking regions collectively forming an outer surface of the tissue-mimicking regions having a shape that follows a shape of the inner surface of the shell.

4. The apparatus of claim 1, wherein an external surface of the first tissue-mimicking region interfaces with an external surface of the second tissue-mimicking region to form a contiguous portion of an outer surface of the tissue-mimicking regions.

5. The apparatus of claim 1, wherein each of the tissue-mimicking regions has a consistent tissue property throughout the tissue-mimicking region.

6. The apparatus of claim 1, wherein the tissue-mimicking regions are arranged in a stacked configuration selected from the group of: cylindrically stacked, spherically stacked, cylindrically stacked with rounded ends, concentrically stacked, stacked slab, stacked centrally split, and a combination thereof.

7. The apparatus of claim 6, wherein:
the phantom includes a housing; and
the tissue-mimicking regions and the phantom housing have a circular cross-section that facilitates transmission of concentric mechanical waves throughout the tissue-mimicking region.

8. The apparatus of claim 6, wherein:
the phantom includes a housing; and
the tissue-mimicking regions and the phantom housing have cylindrical outer shells that facilitate transmission of concentric mechanical waves throughout the tissue-mimicking region.

9. The apparatus of claim 1, wherein each tissue-mimicking region includes a compartment, with one or more compartments being stacked on a first compartment, and with each compartment being filled with a material having a tissue property that is different from material filling another one of the compartments.

10. The apparatus of claim 1, wherein at least one of the tissue-mimicking regions includes a thermoset material, cross-linked polymeric material, or gel-based material that has a melting point higher than its gel point, and is configured to set in response to being heated and subsequently cooled, therein facilitating the application of another type of material onto a previously formed material while mitigating changes in properties of the previously formed material.

11. The apparatus of claim 1, wherein for each tissue-mimicking region, a spatial extent occupied by the region and materials therein are selected and positioned relative to the other tissue-mimicking regions to facilitate measurement of stiffness of the material in the tissue-mimicking region, based on one or more characteristics of an imaging system selected from the group of: excitation wavelength, frequency, passive driver type and format, and a combination thereof.

12. The apparatus of claim 1, wherein at least one of the tissue-mimicking regions is configured to simultaneously mimic both mechanical stiffness and magnetic resonance imaging (MRI) proton density fat fraction.

13. A method for analyzing an imaging system, the method comprising:
imaging a phantom apparatus having:
a first tissue-mimicking region having a first tissue property; and
at least one additional tissue-mimicking region, including a second tissue-mimicking region having a second tissue property that is different than the first tissue property, the second tissue-mimicking region being stacked on the first tissue-mimicking region; and
analyzing the imaging system based on images of the respective tissue-mimicking regions.

14. The method of claim 13, wherein respective surface portions of each of the tissue-mimicking regions collectively form an outer surface of a portion of the phantom apparatus that consists of the tissue-mimicking regions, each tissue-mimicking region having such a surface portion that is contiguous with such a surface portion of another of the tissue-mimicking regions.

15. The method of claim 13, wherein:
the tissue-mimicking regions are arranged in a configuration selected from the group of: a stacked configuration, a concentric configuration, a centrally split configuration, and a combination thereof; and
analyzing the imaging system includes generating images or a measurement of each region.

16. The method of claim 13, wherein the tissue-mimicking regions are stacked on one another, and analyzing the imaging system includes assessing mechanical stiffness of each tissue-mimicking region using a multi-slice magnetic resonance elastography (MRE) approach in which one or more slices lie in a stacked region that is different than a stacked region in which another one of the slices lies.

17. A method of manufacturing a phantom, the method comprising:
forming a first tissue-mimicking region having a first tissue property; and
forming at least one additional tissue-mimicking region, including a second tissue-mimicking region having a second tissue property that is different than the first tissue property, the second tissue-mimicking region being stacked on the first tissue-mimicking region.

18. The method of claim 17, wherein forming the tissue-mimicking regions includes forming respective surface portions of each of the tissue-mimicking regions to collectively form an outer surface of a portion of the phantom apparatus that consists of the tissue-mimicking regions, each tissue-mimicking region having such a surface portion that is contiguous with such a surface portion of another of the tissue-mimicking regions.

19. The method of claim 17, wherein forming the phantom includes arranging the regions in a configuration selected from the group of: a stacked configuration, a concentric configuration, a centrally split configuration, and a combination thereof.

20. The method of claim 17, wherein forming the phantom includes forming each region with a spatial extent and materials selected and positioned relative to the other regions to facilitate measurement of stiffness of the material in the region, based on one or more characteristics of an imaging system selected from the group of: excitation wavelength, frequency, passive driver type and format, and a combination thereof.
